SEQUENCE LISTING (3 sequences)

SEQ ID NO: 1 - DNA, 10 bp, Artificial Sequence
  Synthetic oligonucleotide
  modified_base (1)..(1): Biotin
  modified_base (10)..(10): amine
aaaaaaaaaa

SEQ ID NO: 2 - DNA, 40 bp, Artificial Sequence
  Synthetic oligonucleotide
  modified_base (40)..(40): amine
tgcgacctcagactccggtggaatgaaggaaaaaaaaaaa

SEQ ID NO: 3 - DNA, 30 bp, Artificial Sequence
  Synthetic oligonucleotide
  modified_base (30)..(30): biotin
tccttcattccaccggagtctgaggtcgca

(19) United States
(12) Patent Application Publication
(10) Pub. No.: US 2018/0264466 A1
(43) Pub. Date: Sep. 20, 2018

(54) POSITION-DEFINED CELL CULTURE AND CHARACTERIZATION PLATFORM

(71) Applicant: The Regents of the University of California, Oakland, CA (US)

(72) Inventors: Yu-Hwa Lo, San Diego, CA (US); Yu Jui (Roger) Chiu, San Diego, CA (US); Wei Cai, San Diego, CA (US)

(73) Assignee: The Regents of the University of California, Oakland, CA (US)

(21) Appl. No.: 15/542,306

(22) PCT Filed: Jan. 8, 2016
(86) PCT No.: PCT/US16/12736

§ 371 Date: Jul. 7, 2017

Related U.S. Application Data
(60) Provisional application No. 62/101,286, filed on Jan. 8, 2015.

(51) Int. Cl. B01L 3/00; C12M 1/00; G01N 33/543; B01J 19/00; G01N 33/569

(52) U.S. Cl. CPC B01L 3/5085; C12M 47/04; G01N 33/54366; B01J 19/0046; G01N 33/56966; B01J 2219/00585; B01J 2219/00612; B01J 2219/00619; B01J 2219/00722; B01J 2219/00725; B01J 2219/00168; B01J 2219/00623; B01J 2219/00317

(57) ABSTRACT

Methods, systems, and devices are disclosed for culturing and characterizing individual cellular entities including cells organoids, or tissue. In one aspect, a device includes a first substrate structured to include an array of hydrophilic regions surrounded by a hydrophobic surface including nanostructures protruding from the hydrophobic surface, in which the array of hydrophilic regions are capable to adhere an individual cellular entity and the hydrophobic surface is configured to prevent the cellular entity from adherence; and a second substrate including a coating of antibodies corresponding to a type of cellular substance secreted by the cellular entity, in which the second substrate is operable to be placed upon the first substrate such that the coating of antibodies makes contact with the individual cellular entities adhered to the hydrophilic regions.

### PRIORITY CLAIM AND RELATED APPLICATION

This application claims the benefits and priority of U.S. Provisional Application No. 62/101,286 entitled “POSITION-DEFINED CELL CULTURE AND CHARACTERIZATION PLATFORM” filed on Jan. 8, 2015, the entire disclosure of which is incorporated by reference as part of this application.

### STATEMENT REGARDING FEDERALLY SPONSORED RESEARCH OR DEVELOPMENT

This invention was made with government support under grant 5R21GM107977 awarded by the National Institutes of Health (NIH). The government has certain rights in the invention.

### TECHNICAL FIELD

This patent document relates to systems, devices, and processes for fabrication and use of nanomaterials in cell culture and characterizations.

### BACKGROUND

Single-cell analysis includes the characterization of individual cells, structurally and functionally, in isolation. The analysis of single cells can provide information about individual cells of a cell population, including cell-to-cell differences otherwise lost in averaged bulk cell measurements. Such information can be of critical importance in the understanding of cellular biological processes and mechanisms, e.g., including gene expression variations, and disease, e.g., including drug resistance in cancer cells.

### SUMMARY

Techniques, systems, and devices are disclosed for cell, tissue, and organoid culture and time-lapsed bioactivity characterizations. The disclosed technology includes a cell and tissue culture and characterization platform including an engineered single-cell placement template device that can be placed in each well of a well plate; and a bioprinting device to monitor single-entity bioactivities by sampling and collecting secretions with high spatiotemporal resolution.

In one aspect, a device includes a first substrate structured to include an array of hydrophilic regions surrounded by a hydrophobic surface including nanostructures protruding from the hydrophobic surface, in which the array of hydrophilic regions are capable to adhere an individual cellular entity and the hydrophobic surface is configured to prevent the cellular entity from adherence; and a second substrate including a coating of antibodies corresponding to a type of cellular substance secreted by the cellular entity, in which the second substrate is operable to be positioned on the first substrate such that the coating of antibodies of the second substrate makes contact with the individual cellular entities adhered to the hydrophilic regions of the first substrate.

The subject matter described in this patent document can be implemented in specific ways that provide one or more of the following features. For example, in some embodiments of the disclosed platform, the template device can include an array of micro-islands with hydrophilic surfaces over a super-hydrophobic surface. The hydrophilic surface of micro-islands favors cell adhesion and can be structured to include a layer of SiO₂ or biocompatible material, e.g., such as a layer of Au or Ti. On the surface of the hydrophilic layer, an extracellular matrix or a cell seedling layer may be applied to improve single-entity adhesion. The surrounding super-hydrophobic surface can include black silicon structured to include vertical nanostructures (e.g., “nanoposts”), which may be configured to be 4-6 micrometers long and <500 nm wide. In some implementations, the black silicon can be used as a mold to transfer its nanopattem and super-hydrophobicity to other materials using soft lithography or hot embossing process. For example, the pattern can be transferred to polydimethylsiloxane (PDMS), poly(methyl methacrylate) (PMMA), and Cyclic Olefin Copolymer to make transparent smart templates for use with inverted microscopes. The bioprinting device can include a glass plate coated with specific antibodies for certain types of cell secretion of interest. When the plate is placed on top of the exemplary template device with a small gap, over time it can capture the secreted component from each single-entity at the designated position. In implementations, for example, the relative position between fiducial marks on the glass plate and the fiducial patterns on the exemplary template device, e.g., recorded through a microscope, allows easy tracking of the secretions over the time period of the bioactivity study. In some embodiments, for example, electrodes can be formed over the hydrophilic areas to enable biosensing, apply voltage to use electrophoretic and/or dielectrophoretic effects to attract (or repel) cells, or for electroporation to introduce drug or macromolecules the cells. The disclosed technology can be utilized to solve critical bottlenecks impeding the utility and impact of single-cell characterizations of importance to biomedical applications in the areas of drug discovery and biological research.

In another aspect, a device for characterization of single cellular entities includes a first substrate structured to include an array of hydrophilic regions surrounded by a hydrophobic surface including nanostructures protruding from the hydrophobic surface. The array of hydrophilic regions is to adhere an individual cellular entity and the hydrophobic surface is to prevent the cellular entity from adherence. The device includes a second substrate including a coating of antibodies corresponding to a type of cellular substance secreted by the cellular entity. The second substrate is positioned on the first substrate such that the coating of antibodies of the second substrate makes contact with the individual cellular entities adhered to the hydrophilic regions of the first substrate.

The disclosed device can be implemented in various ways to include one or more of the following features. For examples, the cellular entity can include a cell, an organoid, or a tissue. The first substrate can be shaped to insert within a well of a multi-well plate. The multi-well plate can include a 96-well plate. The first substrate can include 1,000 or less hydrophilic regions in the array. The hydrophilic regions of the array can include a dimension in a range of 50 μm to 400 μm. The hydrophilic regions can include silicon oxide (SiO₂). The hydrophilic regions can include a biocompatible material. The biocompatible material can include gold (Au) or Titanium (Ti). The hydrophilic regions can be structured to include a layer forming an extracellular matrix (ECM) or a cellular seeding layer. The nanostructures can include vertically aligned nanostructures including one or more of nanopillars, nanoposts, or nanopins having a diameter of 500 nm or less and a height of 6 μm or less. The second substrate can include fiducial markers in an arrangement to provide a point of reference or measurement for an image of the cellular substance bound to the coating. The cellular substance can include exosomes, signaling proteins, or cytokines. The hydrophilic regions can include micro-islands and the hydrophobic surface can include black silicon. The hydrophobic surface can have a contact angle greater than 155 degrees.

In another aspect, a method of fabricating a template for characterization of single cellular entities can include disposing hydrophilic wells over a substrate; and disposing hydrophobic nanostructures surrounding the hydrophilic wells. The hydrophilic wells and the hydrophobic nanostructures can be formed using separate etch masks. The hydrophobic nanostructures can include black silicon. The wells can range from a few micrometers to over 100 μm. The substrate can include a transparent substrate. The substrate can include glass or quartz. The method can include disposing a layer of amorphous Si on the transparent substrate. The substrate can include a silicon-on-sapphire wafer.

### BRIEF DESCRIPTION OF THE DRAWINGS

FIG. 1 shows a schematic illustration of a cell and tissue culture and characterization platform of the disclosed technology including an engineered single-cell placement template device placed in wells of a well plate for cell, tissue, and/or organoid sorting, placement, culturing, and time-lapse studies.

FIG. 2 shows a schematic illustration of an exemplary high throughput cell activity characterization for (a) cell proliferation and phenotypical characteristics, and (b) time lapse analysis using an exemplary bioprinting technique of the disclosed technology.

FIG. 3 shows schematic illustrations and images depicting the structure and properties of an exemplary template device of the disclosed technology.

FIG. 4 shows an image depicting self-registration of cells over the hydrophilic micro-islands surrounded with super-hydrophobic black silicon of the exemplary template device.

FIG. 5 shows a schematic illustration of an exemplary fabrication technique to produce an exemplary template device of the disclosed technology, and an image of the exemplary fabricated template device.

FIG. 6 shows in panel (A) Single MCF7 cell on the hydrophilic microisland after 48 hour culture; in panel (B) SEM micrograph of the exosomes secreted by the MCF7 cell; and in panel (C) Labeling the exosomes with quantum dots to become visible under fluorescent microscope.

FIG. 7 shows an exemplary process flow for recessed hydrophilic islands surrounded with superhydrophobic black silicon for culture and analysis of tissues, organoids, or both.

FIG. 8 shows am exemplary process flow for recessed hydrophilic islands surrounded with superhydrophobic black silicon on a transparent substrate to allow in-situ microscopy during culture.

FIG. 9 shows a structure of an exemplary evaporating droplet array that includes hydrophilic islands surrounded by a superhydrophobic surface.

FIG. 10 shows a schematic of an exemplary DNA/RNA detection: panel (a) amine end-linked probes immobilized to aldehyde-activated microislands; panel (b) sample droplets of streptavidin labeled miRNA mimic oligonucleotides are pipetted onto the hydrophilic islands; panel (c) the concentration of the miRNA mimic oligonucleotides is increased and the volume of the sample droplet is reduced by evaporation; panel (d) oil drop stops the evaporation process and the reaction happens within the encapsulated nanochambers; panel (e) Oil layer and hybridization buffer are washed away; (f) DNA duplex is labeled with QDs for fluorescent detection.

FIG. 11 shows an exemplary fabrication process of the microarray for evaporating droplets; panel (a) mechanical grade silicon wafer is cleaned for microfabrication; panel (b) photoresist NR9-1500 is patterned on the silicon wafer by lithography; panel (c) SiO2 and Cr layers are deposited on the substrate by sputtering; panel (d) a microarray of SiO2/Cr dots is patterned on the silicon wafer; panel (e) nanopillars are etched by the DRIE process; panel (f) the Cr layer is removed by chromium etchant; panel (g) a photograph showing the reflectivity difference between black silicon and islands of SiO₂ covered silicon.

FIG. 12 shows a schematic of an exemplary DNA/RNA detection procedure on the hydrophilic surface; panel (a) The surface is linked to APT; panel (b) the aldehyde group of GTA is bonded to the amino group of APTS; panel (c) The DNA oligonucleotide probe with amine modification at the 3′ end is linked to the GTA; panel (d) Target DNA with biotin modification at the 3′ end is hybridized with the anchored DNA probe; panel (e) streptavidin conjugated quantum dots are bonded to DNA duplex for visualization.

FIG. 13 shows SEM images of nanopillars fabricated using the DRIE method: panel (a) 45° view panel (b) top view panel (c) interface between the hydrophilic island and the nanopillars. All the scale bars in (a, b, c) are 2 micrometer.

FIG. 14 shows the evolution of an evaporating water droplet on SiO₂ patterned black silicon. (panels a,b,c) photographs of droplet at evaporation time of 1 min, 20 min, and 40 min; panel (d) micrograph of droplet at evaporation time of 50 min; panel (e) schematics of the evolving shape of the droplet at 1 (1), 20 (2), 40 (3), 50 (4), and 53 (5) min; panel (f) the contact angle and contact diameter dependence on evaporation time. The scale bars in (panel a, b, c) are 1 mm, and the scale bar in (d) is 200 micrometer.

FIG. 15 shows sample droplet was self-aligned with the SiO₂ island during evaporation: panel (a) 1 min; panel (b) 15 min; panel (c) 35 min; panel (d) 45 min. The scale bars are 400 μm.

FIG. 16 shows panel (a) Bright view image of clean SiO₂ island surrounded by black silicon after microfabrication process; panel (b) Fluorescent image of the FITC labeled DNA dried on the SiO₂ island; panel (c) Detected fluorescence intensity of FITC labeled DNA dried on the SiO₂ island. The scale bars are 200 μm.

FIG. 17 shows a linear relationship was obtained between the detected number of streptavidin-biotin binding and the concentration of streptavidin in the sample solution.

FIG. 18 shows panel (a) The linear dependence of the number of hybridized targets and the concentration of target molecule in the sample; panels (b-d) the processed images of visualized quantum dots with a target concentration of 100 fM, 1 pM, and 10 pM, respectively.

FIG. 19 shows an exemplary high throughput drug screen chip implemented using the disclosed technology.

FIG. 20 shows an exemplary high throughput drug screen—silicon chip based platform implemented using the disclosed technology.

### DETAILED DESCRIPTION

Single-cell analysis holds promise to unveil the underpinnings of biological processes that have evaded detection because single-cell analysis enables sensitive and accurate quantification of single-cell properties amidst biological samples with known, but difficult to quantify, heterogeneity (e.g. cancer stem cells in tumor tissue). However, achieving single-cell analysis relies on advancing several technologies, including single-cell isolation, detection, genetic and proteomic analysis, culture and co-culture, and measurements over time. While significant advances have been achieved in areas such as single-cell transcriptomics and genomics, major road blocks exist in realizing the full potential of single-cell technology. Among these unmet challenges include a throughput bottleneck, high capacity single-cell tracking, and time-lapse analysis of single-cell activity.

To pinpoint elusive cellular processes in biomedicine, e.g., such as drug resistance, cancer development, and immunology, scientists need to analyze biological samples at single-cell resolution, to reveal the exceptional properties of rare cells that are masked by population averaging. However, skepticism still exists regarding how much biomedical insight can be actually obtained from single-cell experiments. Researchers have demonstrated single-cell genomics, proteomics, and transcriptomics, but other than showcase technology, the biological insight and clinical utility of single-cell technologies have not yet lived up to their promise. Unless one can analyze the properties of thousands or tens of thousands of cells within a reasonably short time period and at a cost not significantly greater than analyzing the ensemble as a whole, the impact of single-cell technology will continue to be limited.

Disclosed are techniques, systems, and devices for a highly efficient single-entity (e.g., cell, tissue, and organoid) culture and analysis platform with engineered materials and devices capable of high throughput, tracking, and time lapse characterization capabilities.

The disclosed single-cell, organoid, and tissue culture and characterization platform includes an engineered single-cell placement template device that can be placed in each well of a well plate; and a bioprinting device to monitor single-entity bioactivities by sampling and collecting secretions with high spatiotemporal resolution. The disclosed technology can be utilized to solve critical bottlenecks impeding the utility and impact of single-cell characterizations of importance to biomedical applications in the areas of drug discovery and biological research.

In some embodiments of the disclosed platform, the template device can include an array of micro-islands with hydrophilic surfaces over a super-hydrophobic surface. The hydrophilic surface of micro-islands favors cell adhesion and can be structured to include a layer of SiO₂ or biocompatible material, e.g., such as a layer of Au or Ti. On the surface of the hydrophilic layer, an extracellular matrix (ECM) or a cell seedling layer (e.g., fibroblast) may be applied to improve single-entity adhesion (e.g., single cells, organoids, or cell tissues). The surrounding super-hydrophobic surface can include black silicon structured to include vertical nanostructures (e.g., “nanoposts”), which may be configured to be 4-6 micrometers long and <500 nm wide. The bioprinting device can include a substrate (e.g., a glass plate) coated with specific antibodies for certain types of cell secretion of interest. In some embodiments, the bioprinting device can include one or more fiducial markers positioned on its substrate, e.g., to be placed in a field of view of an imaging system (e.g., microscope), to provide a point of reference or a measure of an image to be produced. When the plate is placed on top of the exemplary template device with a small gap, over time it can capture the secreted component from each single-entity at the designated position. In implementations, for example, the relative position between fiducial marks on the glass plate and the fiducial patterns on the exemplary template device, e.g., recorded through a microscope, allows easy tracking of the secretions over the time period of the bioactivity study. In some embodiments, for example, electrodes can be formed over the hydrophilic areas to enable biosensing, apply voltage to use electrophoretic and/or dielectrophoretic effects to attract (or repel) cells, or for electroporation to introduce drug or macromolecules the cells.

In some implementations, the vertical nanostructures of the black silicon super-hydrophobic surface can be formed with mask or patterning using laser assisted or plasma etch. For example, alternating the plasma etching process and the surface passivation process with SF6 and C4F8 gases in a plasma chamber can turn a regular Si wafer into black silicon, so named for its black appearance due to the strong photon trapping effect to prevent visible light from escape. The exemplary black silicon surface provides a super-hydrophobic property, e.g., in which exemplary characterizations of the surface showed that a contact angle of water droplet on the black silicon surface can be greater than 155 degrees, making the surface especially hard for biological cells to adhere to.

In some implementations, the black silicon can be used as a mold to transfer its nanopattern and super-hydrophobicity to other materials using soft lithography or hot embossing process. For example, the pattern can be transferred to polydimethylsiloxane (PDMS), poly(methyl methacrylate) (PMMA), and Cyclic Olefin Copolymer (COC).

Although these exemplary polymer materials are transparent so the finished materials do not look black, they contain the nanoscaled features of black silicon mold and possess super-hydrophobicity. For some applications, for example, making such templates devices out of such transparent materials is attractive because it allows microscopy using an inverted microscope without disturbing cell growth.

By placing the exemplary template device in the wells of a well plate (e.g., such as a 96-well plate), one can dispense different cell types (e.g., presorted cells or directly from cell mixtures) into of the 96 wells, and the cells in each well precipitate onto the hydrophilic micro-islands, such that very few (e.g., <1%) cells staying outside the micro-islands and on the super-hydrophobic surface. In this manner, the cell positions are well defined and spatially confined during culture, to allow continuous observation and analysis of the same cells in the same location without losing track of them.

For example, in the course of cell growth, one may not only monitor the morphological and phenotypical properties using a microscope but also perform molecular analysis of cells without disturbing them. For molecular analysis, for example, a glass plate with fiducial markers coated with specific anti-bodies can be placed on top of the exemplary template device with a small gap to avoid physical contact. Over a period of time (e.g., typically between 10 minutes and 2 hours depending on the secretion rate of molecules being measured), the molecules secreted by the cells on the micro-islands are captured by the glass slide. From the glass slide one can collect the specific types of molecules secreted by the cells at the corresponding positions. The method offers high-throughput, quantitative bioactivity analysis of individual cells.

For example, exosomes are vesicles secreted by cells, and it is generally believed that cancer cells have higher exosome secretion rates than normal, non-proliferating cells. However, there exists no method today for quantitative measurement of exosome secretion rates for different cell types and for different individual cells under different microenvironments, culture conditions, and stimulations such as mechanical stress, drugs, and toxins. The disclosed technology includes methods that quantify exosome secretion rates from different cell types with single-cell resolution. In some implementations, for example, CD63 immobilized glass plates can be used to capture exosomes secreted by specific cells at designated locations and specific moments. After exosome collection, miRNAs or proteins can be extracted from the exosomes and molecular analysis is performed to obtain vital biological information related to diseases and development of therapy. The same principle can also be applied using the disclosed technology to analyze cytokine secretion from specific cells by simply changing the antibodies or capturing molecules on the plates. Examples include, but are not limited to, vasopressin antibody to capture vasopressin, anti-tyrosine antibody to capture tyrosine hydroxylase, phosphor-Troponin I to capture phosphorylated cardiac troponin-I, β1-AR antibody to capture β1 adrenergic receptor, TATP2A2/SERCA2 to capture SERCA2, among others.

Single-Cell Isolation, Placement, and Culture

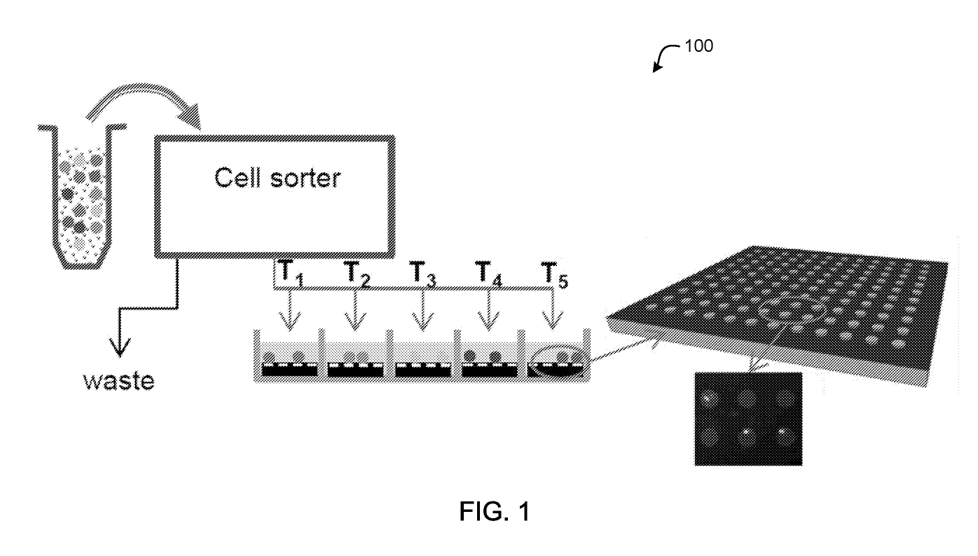

FIG. 1 shows a schematic illustration of an exemplary system 100 for high-throughput single-cell isolation, placement, and culture. Heterogeneous cell suspensions are first sorted with a flow cytometer/cell-sorter to place cells of interest into wells of a standard multi-well plate. For example, the exemplary system 100 can sort, place, and culture cells to wells of a 96-well plate, sequentially, in which within each well includes a microfabricated cell plate of the disclosed technology including a large array of hydrophilic regions (e.g., ‘microislands’) surrounded by super-hydrophobic surface (e.g., black silicon) to anchor the cells in designated positions for culturing and time-lapse studies. The hydrophilic microislands are a silicon (Si) substrate coated with a layer of silicon oxide (SiO2) or with an extracellular matrix (ECM) network or a seed layer of fibroblasts. The dimensions of the microislands are lithographically defined, typically ranging from 50 μm to 400 μm in diameter depending on the application (e.g. cell types of interest). In a few hours, the sorted cells become adherent to the hydrophilic islands with no or few cells (<1%) over the super-hydrophobic area. In this manner, every cell of the same class (based on the cell sorting criteria) finds its position on the single-cell positioning device, This process is repeated in 96 wells, to produce a high-throughput and efficient process with the advantages that: (a) one can set different culture conditions (media, treatments, etc.) across wells; (b) all cultures can be performed in an otherwise conventional culture format without perfusion and metabolic waste removal issues of microfluidic devices.

Single-Cell Time-Lapse Characterization Using a Bioprinting Method

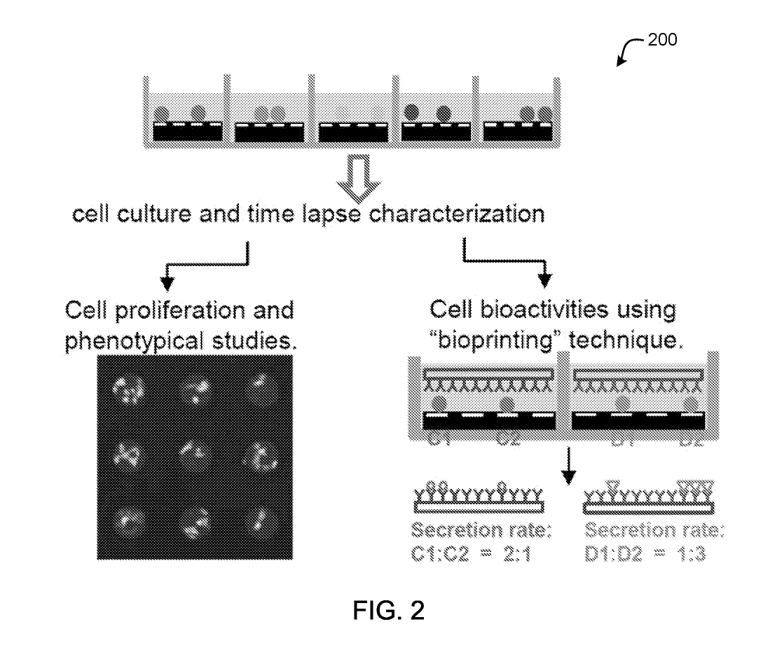

FIG. 2 shows a schematic illustration of an exemplary high throughput cell activity characterization process 200 for (a) cell proliferation and phenotypical characteristics, and (b) time lapse analysis using an exemplary bioprinting technique of the disclosed technology, which can study the single cell secretion rates for exosomes, cytokines, etc. FIG. 2 shows the work-flow of high-throughput time-lapse characterization of a large number of single cells. Conventional microscopy monitors the single-cell morphology over time. Routine microscopy is enhanced by well-defined single-cell locations using the single-cell positioning device to allow efficient cell tracking. Several site-visiting automated microscopy solutions exist to capture these data over time.

The disclosed bioprinting technique allows for time-lapse analysis of single-cell activities such as exosome or protein secretion. Thus, the disclosed technology provides quantitative methods to measure the secretion rate of cells at the single-cell level. Using the disclosed technology, it is possible to compare the secretion rate between different cell types without a reliable reference to normalize the measured results.

The bioprinting plate is simply a glass substrate coated with specific antibodies for certain types of cell secretion of interest. When the plate is placed on top of the cell-positioning device with a small gap (typically 500 μm) over a defined period of time (e.g. 10 minutes to 2 hours), the plate can capture the secreted component from each single-cell at the designated position. The relative position between the fiducial markers on the glass plate and the fiducial patterns on the cell plate, recorded with a picture through a low magnification (5×) microscope, allow easy tracking of the amounts of secretion from the corresponding cells over the time period of study, typically ranging from 2-21 days.

For example, by bioprinting a glass plate with a CD63 coating, the plate can capture exosomes secreted from each cell. Afterwards the collected exosomes on the plate can be detected with a labeled secondary antibody for high-throughput analysis of secretion rate from each cell. Similarly, the bioprinting plate can be coated with antibodies for signaling proteins or cytokines (e.g. CA 15-3 and HER-2/neu for breast cancer, Troponin, CD45, interleukin, TNFα, Interferon gamma, etc.) to study cytokine secretion rates of single-cells. For exosomes, one can further extract the miRNAs and proteins within the collected exosomes from single-cells for sequencing (e.g. miRNA sequencing) or microarray analysis, thus greatly enhancing the amount of relevant information, the measurement accuracy, and the throughput of single-cell technologies.

The disclosed technology platform and assay can include two components: (a) an innovative single-cell placement device placed in each well; and (b) a bioprinting device to monitor single-cell bioactivities by sampling and collecting cell secretions with high spatiotemporal resolution. The technology holds great promise to solving the most critical technology bottleneck that has limited the utility and impact of single-cell studies on which the biomedical community has put so much hope and investment.

Cell-Placement Device Guided by Surface Properties

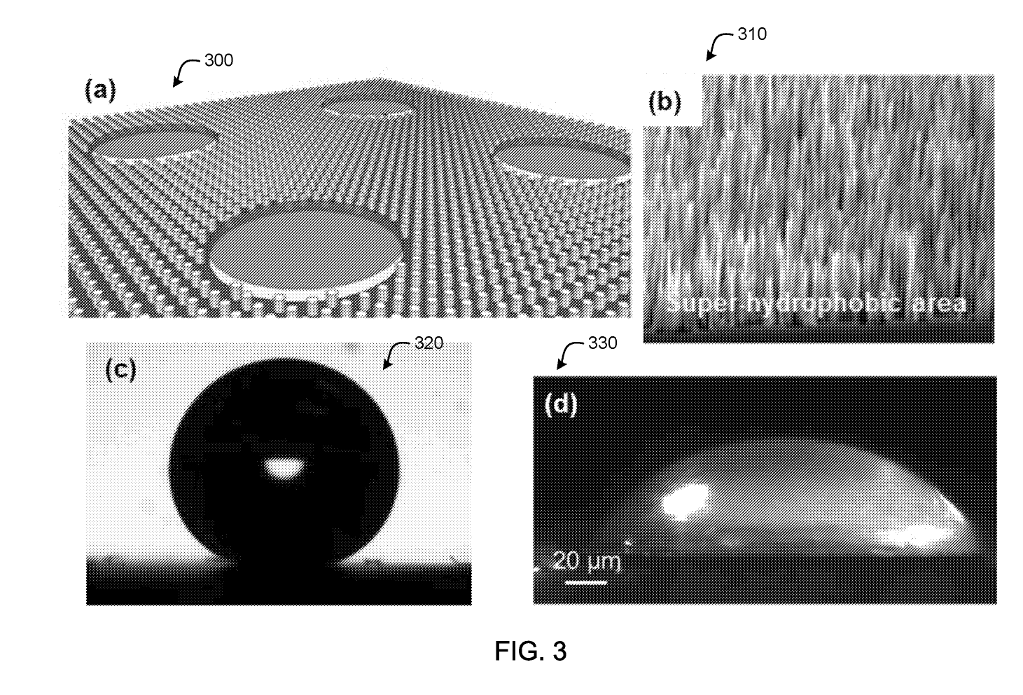

FIG. 3 shows schematic illustrations and images depicting the structure and properties of an exemplary template device of the disclosed technology. FIG. 3 panel (a) 300 shows a schematic illustration showing an exemplary array of hydrophilic islands surrounded with super-hydrophobic surface of black silicon. FIG. 3 panel (b) 310 shows a scanning electron microscopy (SEM) image of the self-formed nanopillar structure of black silicon. FIG. 3 panel (c) 320 shows an image of a contact angle measurement of water droplet on black silicon, which depicts the characteristics of super-hydrophobicity (e.g., contact angle>150 degrees). FIG. 3 panel (d) 330 shows an image of a contact angle measurement of water droplet on the hydrophilic island (e.g., 200 μm diameter).

FIG. 3 shows the fabricated device and characteristics of the single-cell positioning device to be placed in each well of a 96-well plate. A black silicon surface that has nanopillar structures is formed by alternating cycles of plasma etch (SF6) and passivation (C4F8). The nanostructures (referred to as grass or nanopillars) of black silicon give rise to its super-hydrophobic properties illustrated by the large contact angle of >155 degrees (FIG. 3, panel (c)).

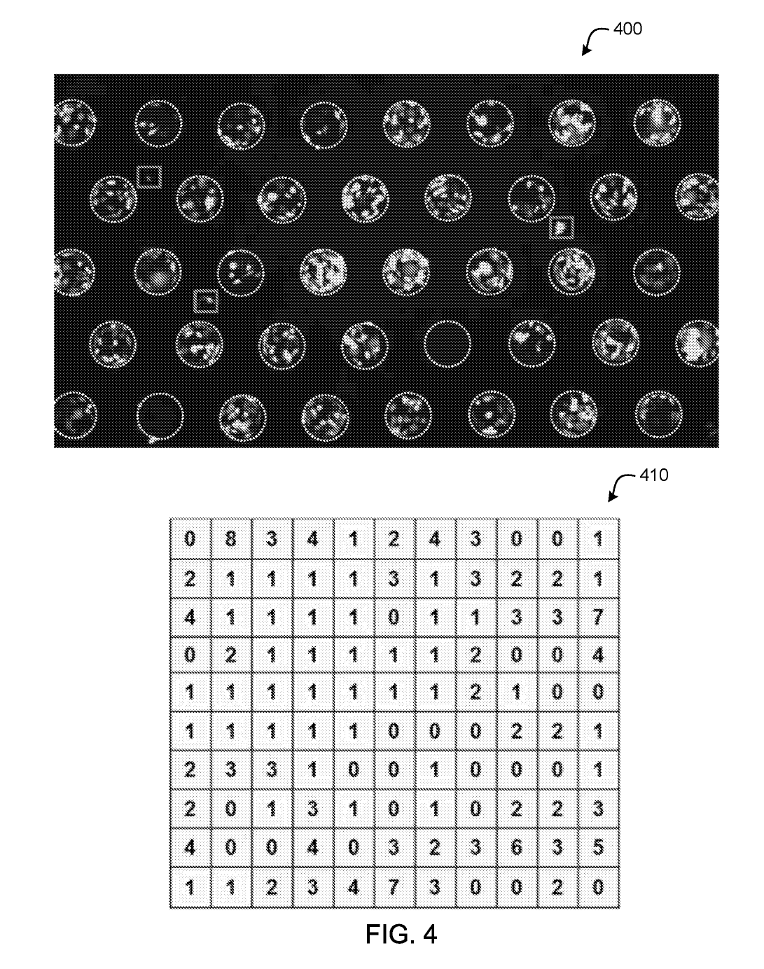

The ability for the device to guide single cells to hydrophilic locations is demonstrated in FIG. 4. FIG. 4 shows an image 400 depicting self-registration of cells (e.g., GFP-transfected MCF7) over the exemplary hydrophilic micro-islands surrounded with super-hydrophobic black silicon. Out of over 7000 cells, less than 1% cells (marked in red boxes) fall outside the micro-islands.

FIG. 4 shows the effectiveness for the proposed device to guide the cell locations. In FIG. 4(a), ˜7×103 cells (GFP transfected MCF7 cells) are placed on the device. After 24 hours of culture, ˜99% of the cells are located in the hydrophilic areas, and only few cells are found on the hydrophobic black silicon surface (those few cells are highlighted in FIG. 4a) even though the black silicon covers around 90% of the device area. FIG. 4b shows the distribution of 200 cells over the device area. Around 37% of the hydrophobic islands are populated by single-cells, which is quite effective for high-throughput single-cell analysis.

The single-cell population does not follow Poisson distribution, suggesting possible effects of surface energies to guide cell motions over the device surface.

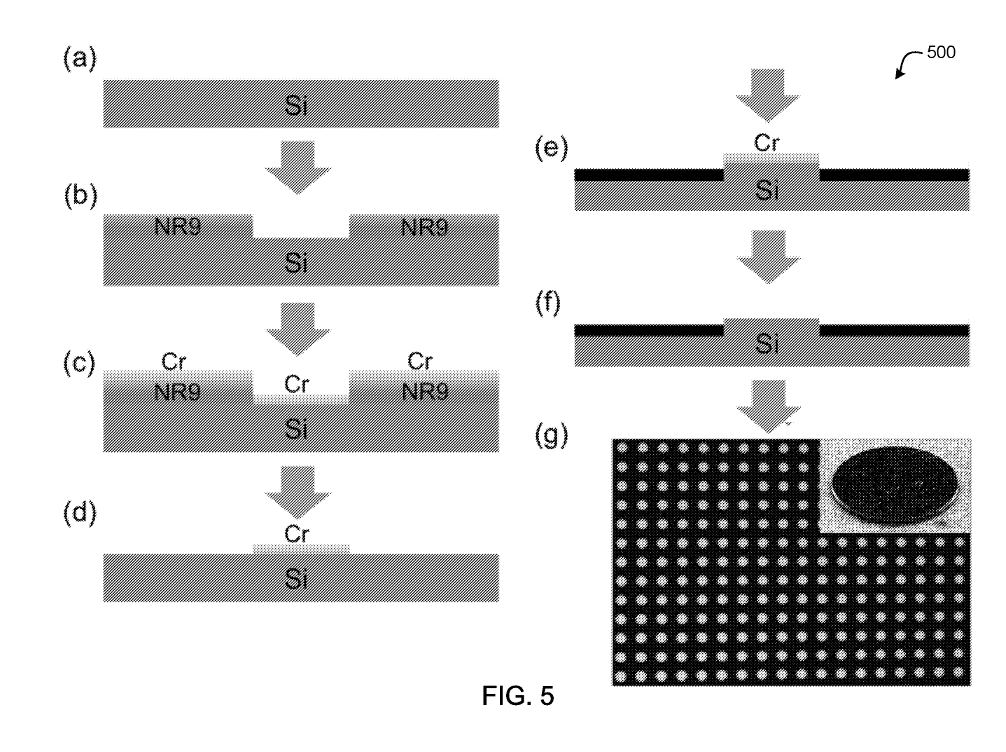

FIG. 5 shows a schematic illustration (in panels (a) to (f)) of an exemplary fabrication technique 500 to produce an exemplary template device of the disclosed technology. FIG. 5 panel (g) shows a photograph of the exemplary fabricated template device with a detailed view and an overall view (inset).

The single-cell placement template has an array of hydrophilic micro-islands surrounded with super-hydrophobic black silicon fabricated on a commercial Si wafer. To form black silicon, the Bosch etching process will be employed in a reactive ion etcher. The etch process consists of alternating cycles of etching (SF6) and passivation (C4F8) to form nanopillar structures. For areas that are protected by lithographically defined SiO2 patterns, no etching or passivation occurs so the hydrophilic properties are preserved after the black silicon process. The process flow is shown in FIG. 5 (panels (a-f)) and FIG. 5 (panel (g)) shows photographs of the finished devices, consisting of an array of 50-400 um diameter hydrophilic islands separated by the super-hydrophobic black silicon surface. We pay particular attention to preserve the super-hydrophobic properties of black silicon in high protein content culture medium. We also monitor closely any trace metals that could be released from black silicon to harm the cells. Due to the enormous surface area of the nanopillar structure of black silicon, release of chemicals from the black silicon surface over time should be investigated. We perform surface Auger spectroscopy and XPS analysis to measure any chemicals on the black silicon surface and identify any potential interference (e.g. heavy metal) with the experimental biology. For instance, the process in FIG. 5 uses chromium (Cr) as etch mask for black silicon formation. Although Cr is not etched by C4F8 plasma, some Cr may be sputtered and stay in the nanostructure of black silicon. Cr could cause adverse chronic effect on cultured tissues and organoids, but can become poisonous to single cells.

To enable time-lapse analysis of the bioactivities of single cells, we develop the bioprinting techniques and protocols for single-cell analysis without disturbing cell growth or tracking. A simple, low-cost process is developed to extract single-cell secretions and its molecular fingerprints.

Demonstration of Time-Lapse Single-Cell Study from Secretion of Exosomes and Cytokines

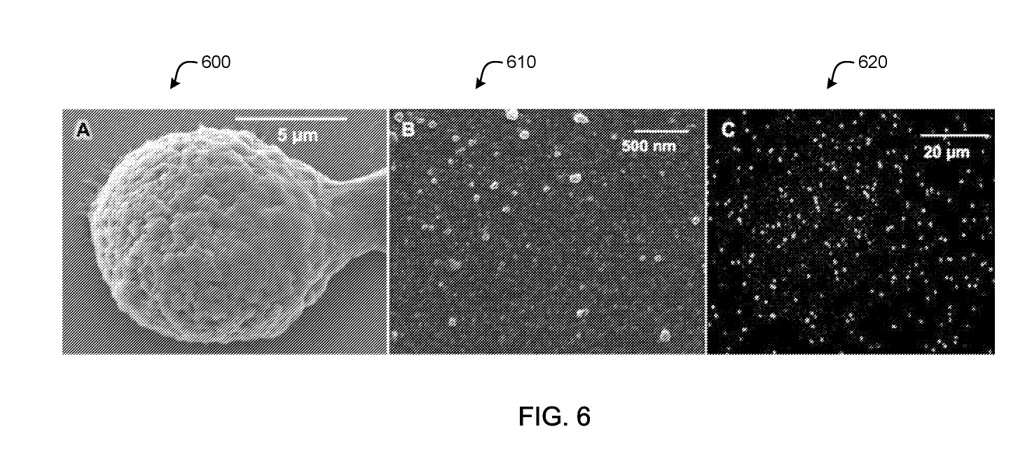

Exosomes are vesicles secreted by cells and it is generally believed that cancer cells have higher exosome secretion rates than normal, non-proliferating cells. However, there exists no method today for quantitative measurement of exosome secretion rates for different cell types and for different individual cells under different environments, culture conditions, and stimulations such as mechanical stress, drugs, and toxins. The invented technology enables us to quantify exosome secretion rates from different cell types with single-cell resolution as we use CD63 immobilized glass plates to capture exosomes secreted by specific cells at designated locations and specific moments. FIG. 6 shows images 600, 610, and 620 of single MCF7 cell on the hydrophilic microisland after 48 hour culture. FIG. 6, panel (A) 600 shows the SEM micrograph of a single-cell on the hydrophilic microisland, and FIG. 6, panel (B) 610 shows the secreted exosomes by the cell. After quantum dot labelling, the exosomes can be visualized under optical microscopy (FIG. 6, panel (C) 620).

To create the antibody coated glass slide, the glass substrate is silanized with 4% solution of (3-mercaptopropyl) trimethoxysilane in ethanol. After washed with ethanol and baked at 100° C., the glass will be immersed in sulfo-GMBS (N-[γ-maleimidobutyryloxy]sulfosuccinimide ester) ethanol solution before being treated with anti-CD63 antibody in PBS solution for 45 minutes at 4° C., followed by BSA treatment to suppress non-specific binding. To attach reporting Q-dots on the collected exosomes, the glass slide will be incubated in biotinylated anti-CD63 antibody (in PBS that contains 1% BSA and 0.09% NaN3) so streptavidin conjugated quantum dots can be linked to the biotinylated exosomes for easy visualization.

The same principle and a similar protocol can also be applied to analyze cytokine secretion from specific cells by simply changing the antibodies or capturing molecules on the glass plates. In this manner, we can investigate CA 15-3 and HER-2/neu, both being breast cancer markers and expected to be secreted by MCF7 cells.

Tissue and Organoid Culture and Analysis

Although single cell analysis reveals information unavailable from cell ensembles, cells are not isolated objects and need to interact and communicate with other cells of the same or different kinds and extracellular structures to function properly. Therefore, culturing cells in forms that best simulate their physiological or pathological conditions provide most relevant information for cell behaviors and responses to drugs and stimuli. The invented platform enables high-throughput, location registered time-lapse studies of not only single cells but also tissues and organoids. An organoid is a three-dimensional organ-bud grown in a laboratory. In a few years since its initial demonstration, scientists have been able to form cerebral organoids, cardiovascular organoids, thyroid organoids, gastric organoids, and intestinal organoids, etc. Because cells in organoids experience similar environments as they live in a complete organ, organoids become popular tools for drug screening and drug response.

The method of placing organoids to the designated positions is similar to the method for single cell placement, except that the hydrophilic islands are usually larger and recessed from the surface of the superhydrophobic black silicon to accommodate the larger size of organoids than single cells.

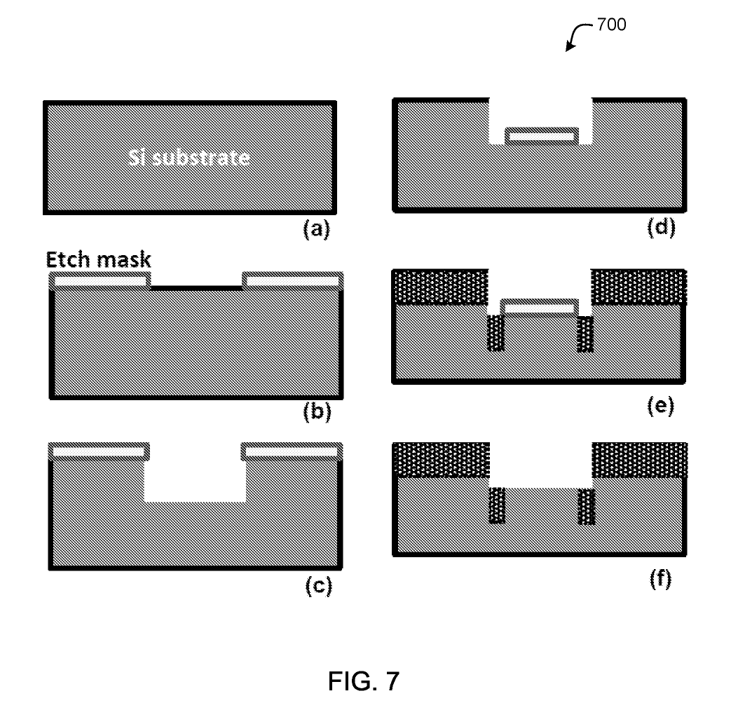

Therefore an exemplary process 700 of template fabrication is different and illustrated in FIG. 7. FIG. 7 shows an exemplary process flow 700 for recessed hydrophilic islands surrounded with superhydrophobic black silicon for culture and analysis of tissues and/or organoids. On the other hand, the technique of bioprinting remains the same as the design for single cell particle secretion analysis.

Photoresist patterns are lithographically defined to form the etch mask for wells that can be formed using dry (plasma) or wet process (FIG. 7, panel (c)). The depth of the wells ranges from a few micrometers to over 100 um depending on the applications. After formation of wells, the photoresist etch mask is removed and a new layer of photoresist pattern is formed as the etch mask for black silicon (FIG. 7, panel (d)). Under the same dry etch process described before, a layer of black silicon is formed surrounding the hydrophilic wells. There may be a ring of black silicon adjacent the recessed hydrophilic wells (FIG. 7, panel (f)) as a result of the process. This black silicon ring does not affect the function of the device.

There can be several variations in the process of forming the surface energy engineered cell registration template, including using other materials than black silicon for the superhydrophobic surface. One particularly interesting design is to form the template on a transparent surface to allow in-situ microscopy using an inverted microscope.

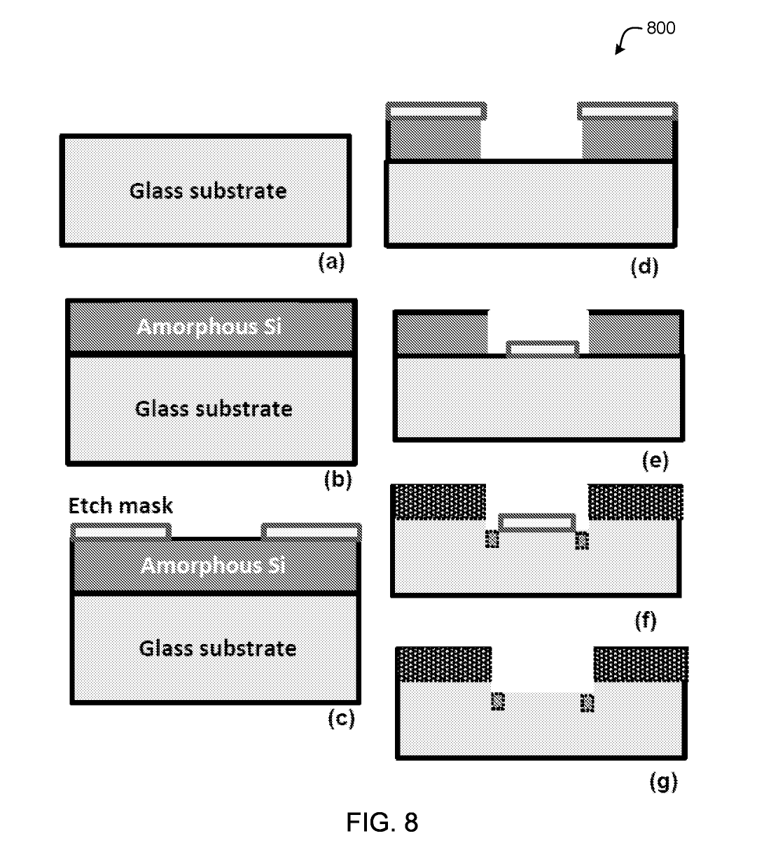

The design of the process 800 is illustrated in FIG. 8. For example, FIG. 8 shows an exemplary process flow 800 for recessed hydrophilic islands surrounded with superhydrophobic black silicon on a transparent substrate to allow in-situ microscopy during culture. The starting substrate is glass or quartz. A layer of amorphous Si is deposited on the transparent substrate using PECVD or sputtering technique. Alternatively, one can use an SOS (silicon-on-sapphire) wafer to achieve Si-on-transparent substrate structure at a higher cost. Another method to form a layer of silicon on a transparent substrate is to use wafer bonding technique. After bonding a silicon wafer to a glass wafer, the Si wafer can be thinned down and chemical-mechanically polished (CMP) to the desired thickness. Once the structure in FIG. 8, panel (b) is achieved, the rest of the process is similar to FIG. 7.

Although black silicon is known to produce excellent superhydrophobic properties to guide the localization of cells and organoids, other materials can also obtain similar characteristics and can be fabricated at lower cost. For example, one can employ the soft lithography method to transfer the nanostructures from black silicon to PDMS which is hydrophobic, transparent, and biocompatible. Or instead of etching, one can use nanoimprinting method to form the nanostructures that give rise to the superhydrophobic properties. Such nanoimprinted structures can be formed using nickel plating electroforming process, followed by hot embossing or injection molding process.

Also one can form electrodes over the hydrophilic areas to enable biosensing (e.g. electrochemical sensing from cyclic voltammetry (CV) or electrochemical impedance spectroscopy (EIS)) or apply AC or DC voltage to use electrophoretic and/or dielectrophoretic effects to attract (or repel) cells. One can also use the electrodes for electroporation to introduce drug or macromolecules such as nucleic acids and proteins to the cells. These features are commonly used in many microfluidic devices, and can be readily integrated with the template to enhance the functionality of the device.

In one embodiment of the disclosed single-cellular entity culture and characterization platform, a device includes a first substrate structured to include an array of hydrophilic regions surrounded by a hydrophobic surface including nanostructures protruding from the hydrophobic surface, in which the array of hydrophilic regions are capable to adhere an individual cellular entity and the hydrophobic surface is configured to prevent the cellular entity from adherence; and a second substrate including a coating of antibodies corresponding to a type of cellular substance secreted by the cellular entity, in which the second substrate is operable to be positioned on the first substrate such that the coating of antibodies of the second substrate makes contact with the individual cellular entities adhered to the hydrophilic regions of the first substrate.

Implementations of the exemplary device include one or more of the following features. For example, the first substrate is shaped to insert within a well of a well plate, e.g., such as within a well of a 96-well plate and including ˜1,000 hydrophilic regions in the array. For example, the hydrophilic regions of the array can include a dimension (e.g., diameter) in a range of 50 μm to 400 μm. For example, the hydrophilic regions can include silicon oxide (SiO2) or a biocompatible material, e.g., such as gold (Au) or Titanium (Ti). For example, the hydrophilic regions are structured to include a layer forming an extracellular matrix (ECM) or a cellular seeding layer (e.g., fibroblasts). For example, the nanostructures can include vertically aligned nanostructures including one or more of nanopillars, nanoposts, or nanopins, e.g., having a diameter of 500 nm or less and a height of 6 μm or less. For example, the second substrate can be configured to include fiducial markers in an arrangement to provide a point of reference or measurement for an image of the cellular substance bound to the coating. For example, the cellular substances to be bound to the antibodies from the individual cellular entities can include exosomes, signaling proteins, or cytokines.

Oil-Encapsulated Nanodroplet Array for Bio-Molecular Detection

Blood and biofluids contain many biomolecules, namely proteins, DNAs, and RNAs that can be used as biomarkers for disease diagnosis. But their low concentration levels often make accurate and rapid detection challenging. For instance, one needs to detect circulating miRNAs at concentrations as low as 10-100 fM for cancers, traumatic brain injuries, cardiovascular diseases, etc. Most of the current surface reaction based biosensors and DNA microarrays have a detection limit of pM, even when the most advanced detection technologies are used (i.e., fluorescence, current or SPR). The detection sensitivity is largely limited by the diffusion process when the concentration of the targets drops to femtomolar range since the flux of diffusion is lowered by the decreasing concentration. The disclosed technology can alleviate the diffusion limit to bridge this performance gap.

While evaporating droplets can be used to enrich target molecules, the detection position and the sensing area are hard to control for reproducible performance. Also the dried DNAs are difficult to identify from the background noise. In a microchip in which the evaporation of DNA droplets took place simultaneously with hybridization, the salt concentration continues to increase with the shrinking volume of the droplet during the hybridization process, which makes the control of hybridization conditions, especially the salt concentration, temperature, and reaction time, rather difficult. The sample may be dried up before the reaction is complete. These factors have limited the detection sensitivity of such device to around 100 pM. Also, a two-stage enrichment device in which the target nucleic acids were first captured by microbeads and then dried for fluorescent detection improved the performance to picomolar sensitivity by separating the molecular enrichment step from the detection step. However, the approach still does not enable precise control of the hybridization conditions to reach the sensitivity required for certain point-of-care in vitro diagnostic applications.

The disclosed technology provides for an oil-encapsulated evaporating droplet array that can detect molecules at a concentration of femtomolar range. Both the detection sensitivity and the reaction speed have been greatly enhanced compared to previous works. The surface properties of the template that supports the droplets have been engineered to allow the droplets to be self-aligned with the sensing areas to facilitate the binding or hybridization process. To further enhance the sensitivity and specificity, a protocol has been developed to have the target enrichment and molecular detection in the same areas of the device without intrachip or interchip sample transfer.

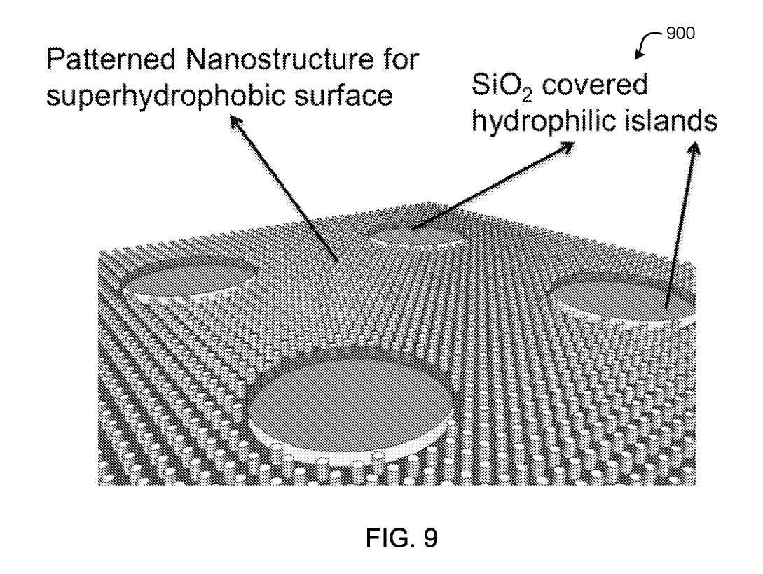

The device 900 includes an array of hydrophilic islands surrounded by a superhydrophobic surface, as shown in FIG. 9. On each hydrophilic island, one type of molecular probes for a specific molecular target can be immobilized. The superhydrophobic nanostructures that surrounds the islands allow the droplet to shrink with a minimal solid/liquid contact area and sample loss. In some implementations, a thin layer of SiO₂ is deposited on the hydrophilic area to attract the sample droplet and to anchor the probes since the SiO₂ surface is compatible with most of the surface modification protocols for biosensors. The SiO₂ islands can have a predetermined size, e.g., a 400 μm diameter and are separated by 4 mm. An array of multiple (e.g., 20) islands is formed on a substrate and, if needed, the design can be easily scaled to have any number of islands and sizes according to the applications. Outside the SiO₂ covered area, nanostructures are formed to be superhydrophobic. For example, the nanostructures turn silicon into black silicon with superhydrophobic properties. The black silicon fabrication process can be adopted as an exemplary process for high throughput and low cost.

Detection of low abundance biomolecules is challenging for biosensors that rely on surface chemical reactions. For surface reaction based biosensors, it require to take hours or even days for biomolecules of diffusivities in the order of 10⁻¹⁰⁻¹¹ m²/s to reach the surface of the sensors by Brownian motion. In addition, often times the repelling Coulomb interactions between the molecules and the probes further defer the binding process, leading to undesirably long detection time for applications such as point-of-care in vitro diagnosis. The disclosed technology can be used to design an oil encapsulated nanodroplet array microchip utilizing evaporation for pre-concentration of the targets to greatly shorten the reaction time and enhance the detection sensitivity. The evaporation process of the droplets is facilitated by the superhydrophilic surface and resulting nanodroplets are encapsulated by oil drops to form stable reaction chamber. Using this method, desirable droplet volumes, concentrations of target molecules, and reaction conditions (salt concentrations, reaction temperature, etc.) in favour of fast and sensitive detection are obtained. A linear response over 2 orders of magnitude in target concentration was achieved at 10 fM for protein targets and 100 fM for miRNA mimic oligonucleotides.

Device Work Flow

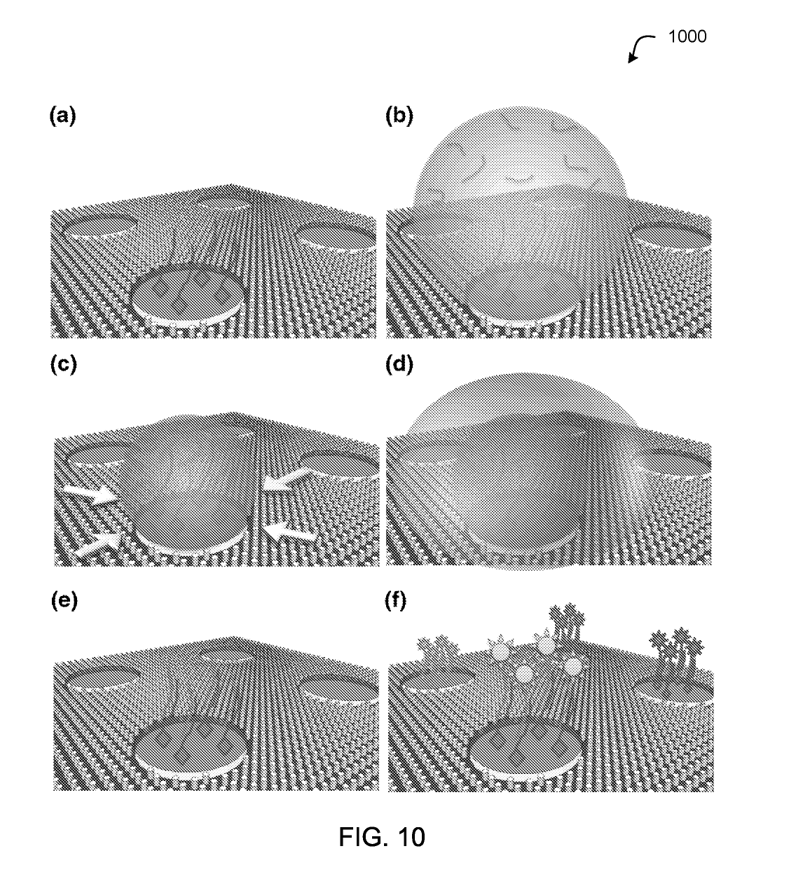

In some implementations, nucleic acid detection 1000 can be used as an example to illustrate the workflow of the biosensor (FIG. 10). In an exemplary detection process 1000, the probes having the complementary sequence to the target nucleic acids were anchored on the SiO2 sensing area. The sample droplets (4 μL each) were dispensed on the template with rough (visual) alignment with the SiO2 islands. By evaporation, the volume of each droplet shrank to 4 nL. Then a layer of oil was dispensed to encapsulate the 4 nL droplets to keep the droplet volume and the salt concentration stable. Within the oil encapsulated nano-chambers, hybridization took place in controlled reaction conditions. The immobilized target bio-molecules were finally visualized and quantified after in situ labelling with streptavidin conjugated quantum dots. The assay was incubated at 50° C. for 30 min or up to 6 h before washing. The length of incubation time showed no obvious effect on detection sensitivity, indicating the diffusion process was not the sensitivity limiting factor within the nanodroplet reactors. The same evaporation droplet process was employed for protein detection with the nucleic acid hybridization process replaced with the protein—ligand binding process.

Device Fabrication

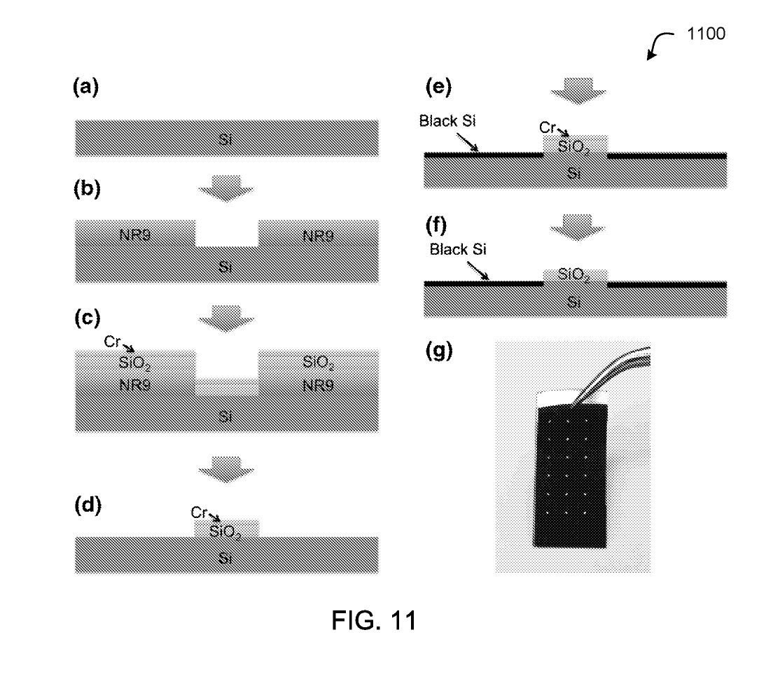

The device according to the disclosed technology can include an array of hydrophilic SiO₂ islands surrounded by a superhydrophilic surface. FIG. 11 shows an exemplary process 1100 for fabricating the device. The array of hydrophilic islands was fabricated using the conventional photolithographic method and nanopillars were formed by deep reactive ion etch (DRIE) over the rest of the Si area to create the black silicon superhydrophobic surface.

The hydrophilic islands were first patterned on a pre-cleaned, mechanical grade silicon wafer by negative tone photoresist NR9-1500PY (Futurrex, USA). FIG. 11, panel (a) shows the silicon wafer substrate. After photoresist patterning (FIG. 11, panel (b)), chromium and SiO₂ films were deposited on the Si wafer using a sputtering system (Denton Discovery 18, Denton Vacuum, LLC) (FIG. 11, panel (c)). The thickness of the Cr/SiO₂ film was 100 nm and 120 nm, respectively. The remaining photoresist was removed by acetone under slight agitation (FIG. 11, panel (d)).

To create a superhydrophobic surface, nanopillars were fabricated using the deep reactive ion etching process. Unlike most top-down process for nanostructure formation that requires definition of nanoscaled patterns and pattern transfer, the nanopillars were formed naturally during the deep reactive etching (Plasmalab System 100, Oxford Instruments) process. In the DRIE process, SF₆ gas was flowed at 30 sccm during the 8 s of reaction time, followed by a passivation cycle when C₄F₈ gas was flowed at 50 sccm for 7 s (FIG. 11, panel (e)). After 80 etching/passivation cycles, dense arrays of nanopillars were formed with an average pillar height of 4.5 μm. During the DRIE process, those islands covered by the Cr layer were protected. In the last step, the Cr layer over the islands was removed by Cr etchant (FIG. 11, panel (f)) to expose the SiO₂ covered islands.

The photograph in FIG. 11, panel (g) shows a device consisting of a 3×6 array of hydrophilic islands. The optical reflectance difference between the array of SiO₂ islands and the surrounding black Si was clearly observed.

Nucleic Acids Detection

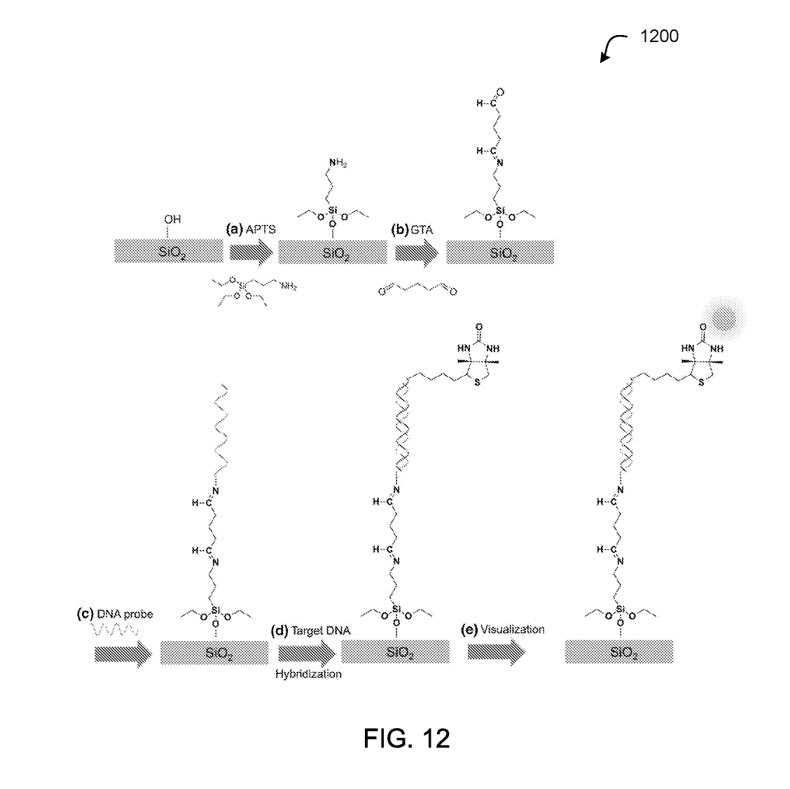

The detection of biomolecules was performed with the above device. FIG. 12 shows an exemplary procedure 1200 to functionalize the SiO₂ surface, immobilize the DNA probe, and detect the target nucleic acids.

At first, aminopropyl-triethoxysilane (APTS) was employed to convert surface silanol group (SiOH) to amine group (NH₂) (FIG. 12, panel (a)). The silicon atom in the APTES molecule formed a chemical bond with the oxygen of the hydroxyl group (OH). Next, glutaraldehyde (GTA) was used as a grafting agent for DNA immobilization. GTA binding was achieved through its aldehyde group (COH) by forming a chemical bond with the amino group of APTES (FIG. 12, panel (b)). For DNA probe immobilization, DNA oligonucleotides with an amine group at 3′ end were linked to the aldehyde group of the linkers (FIG. 12, panel (c)). The target nucleic acids with biotin at 3′ were hybridized with the DNA probe of complementary sequence (FIG. 12, panel (d)). Finally, the amounts of hybridized DNA/RNA or DNA/DNA duplex were quantified by streptavidin conjugated quantum dots (FIG. 12, panel (e)).

Surface Roughness of Black Silicon

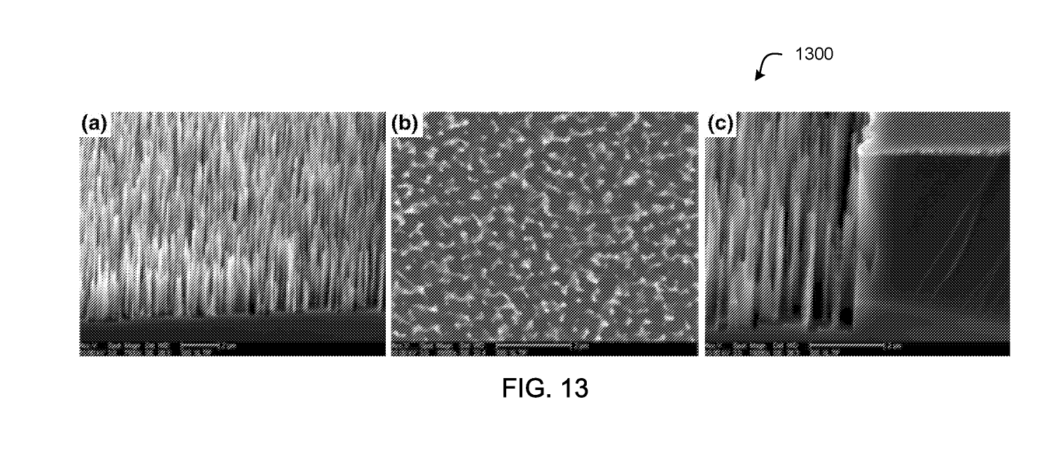

The evaporation process of droplets is significantly influenced by the surface roughness, hydrophobicity and contact angle hysteresis. The surface profile of the SiO₂ patterned black silicon template can be examined using an environmental scanning electron microscope (ESEM, FEI, XL30). FIG. 13 shows images (a), (b), and (c) of nanopillars with around 300 nm diameter, 300 nm spacing and 4.5 μm height. The fluoride coating resulted from the DRIE process and the increased surface roughness produce the superhydrophobic property. The SiO₂ islands are around 1.5 μm higher than the black silicon surface (FIG. 13, image (c)). Minimizing the height difference between the SiO₂ islands and the black silicon surroundings reduces the adhesion of target molecules to the sidewall of the islands while the sample droplet solution shrinks by evaporation.

Contact Angle Measurement

Contact angles of a 4 μL water droplet were measured at 25° C. by the sessile-drop method with a contact-angle goniometer. The values reported here were the averages of three measurements. The same instrument was used to observe evolution of water droplets during evaporation. The contact angle of an evaporating droplet was measured continuously until the droplet was dried. Several droplets were observed during evaporation to assure consistency of the data.

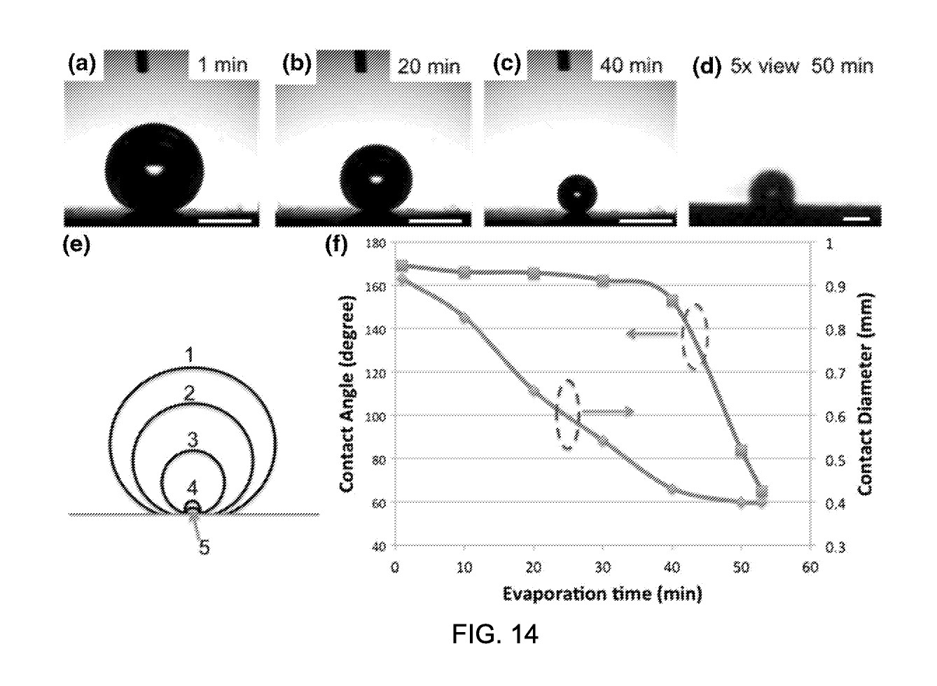

FIGS. 14, panels (a)-(e) show the evolution of water droplets on a patterned black silicon template. The static contact angle is 169.22°, suggesting the superhydrophobic nature of black silicon. Before the droplet shrank towards the 400 μm diameter hydrophilic island, the contact angle of the receding line was approximately constant and the contact diameter decreased steadily (FIG. 14, panel (f)). As soon as the boundary of the droplet reached the SiO₂ island, the contact angle dropped suddenly and the contact line of the droplet was pinned to the boundary of the SiO₂ island.

Self-Alignment Properties of Evaporating Droplet

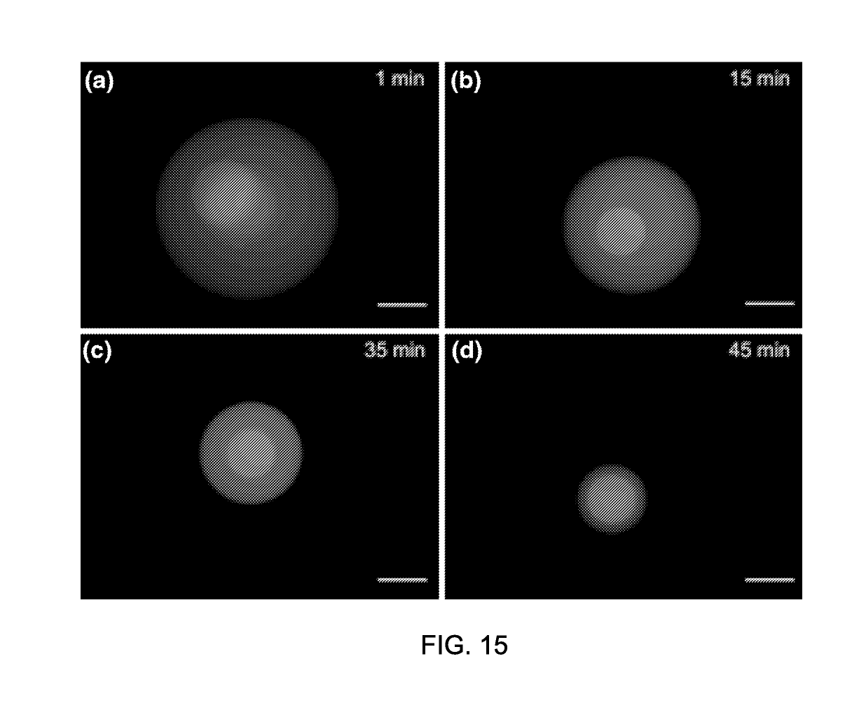

An upright fluorescent microscope (Axio Imager, Zeiss) was used to observe the droplet evaporation over time from the topview. A Xenon acr lamp was mounted on the microscope for illumination. A 4 μL droplet of water with diluted Rhodamine was pipetted onto the patterned black silicon template. Because of the SiO₂ hydrophilic islands, the droplet found a stable area to reside when being dispensed. However, due to the large size mismatch between the droplet and the SiO₂ island, the droplet was often misaligned with the SiO₂ island even though the droplet covered the SiO₂ island. As the evaporation process went on, the droplet shrank towards the center of the SiO₂ island till the contour of the droplet was aligned with the boundary of the SiO₂ island (FIG. 15, images (a), (b), (c), and (d)). By self-alignment, sample droplets can be easily controlled on the black silicon template, which greatly facilitates the droplet dispensing process and molecular sensing process for point-of-care applications.

Fluorescently Labeled DNA Oligonucleotides Concentrated onto the Sensor Area

To test the capability of the evaporating droplets for sample enrichment, a 4 μL droplet of FITC labelled DNA oligonucleotides diluted in distilled water was pipetted on the device. Solutions of progressively decreasing concentration were examined. The droplets were dried at 37° C. and investigated under an inverted epifluorescence microscope (Eclipse TE2000U, Nikon). After background subtraction, the average intensity over the entire SiO₂ island was analyzed using ImageJ and a custom image analysis Matlab program.

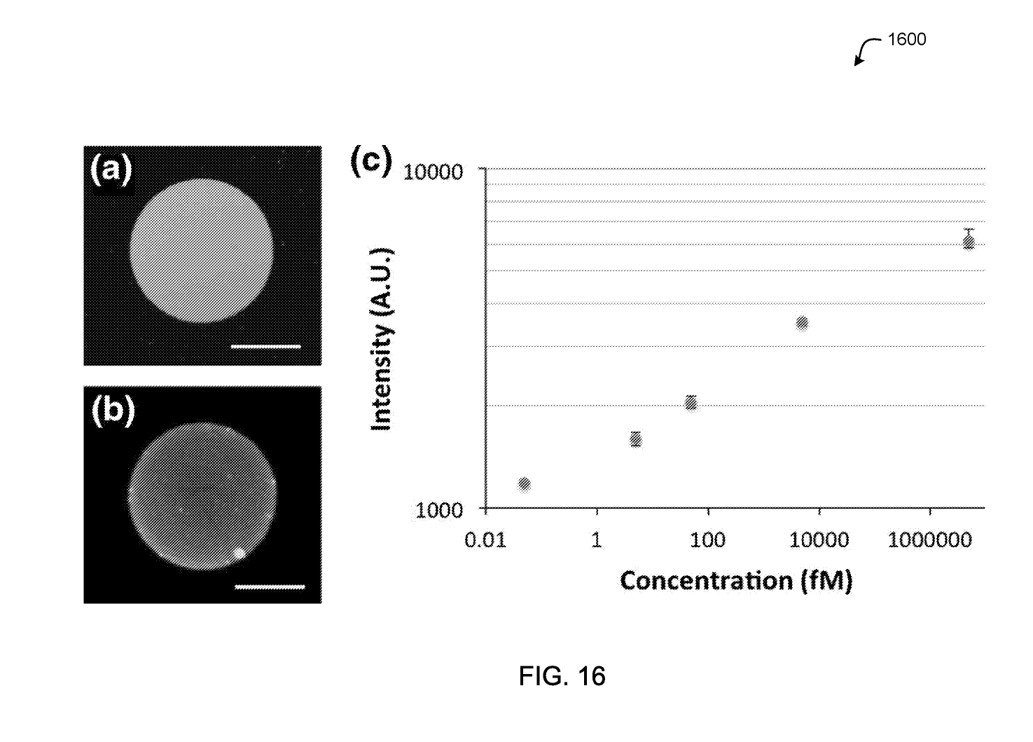

FIG. 16, panel (a) shows a bright view image of clean SiO₂ island surrounded by black silicon after microfabrication process. When the fluorescently labelled DNA oligonucleotides solution was completely dried on the SiO₂ islands, the molecules were uniformly distributed over the entire hydrophilic surface (FIG. 16, panel (b)). The clean background on the black silicon surrounding region suggested that the sample loss due to the liquid/solid boundary movement during evaporation was minimal. As shown in FIG. 16, panel (c), a concentration lower than 50 fM was detectable above the background noise.

Protein Detection

For streptavidin detection, the hydrophilic islands were pre-anchored with biotin-linked DNA oligonucleotides. The DNA oligonucleotides sequence was: 5′-Biotin-AAAAA AAAAA-amine-3′. Target streptavidin was conjugated with quantum dots (Qdot 525, Life technologies) for visualization. Sample droplets (4 μL each) with different concentrations of quantum dots-strepavidin complex were spotted on the black silicon template. The assay was incubated at 37° C. to accelerate the evaporation process. The contact area of the droplet is fixed by the hydrophilic surface of the SiO₂ island, and the height of the droplet was monitored by a goniometer as the droplet volume decreased by evaporation. When the height of the droplet approached the target value, we optically zoomed in by 259 to closely monitor the droplet height. As soon as the sample droplet shrank to 4 nL, a drop of silicone oil (S159-500, Fisher Chemical) was employed to encapsulate the sample droplet and stop the evaporation. The assay was further incubated at room temperature for 1 h before it was dipped in hexane solution to remove the silicone oil. The assay was then cleaned by gentle shaking in TBST buffer and Milli-Q water for 5 and 3 min, respectively. After blowing dry with nitrogen, the assay was ready for observation.

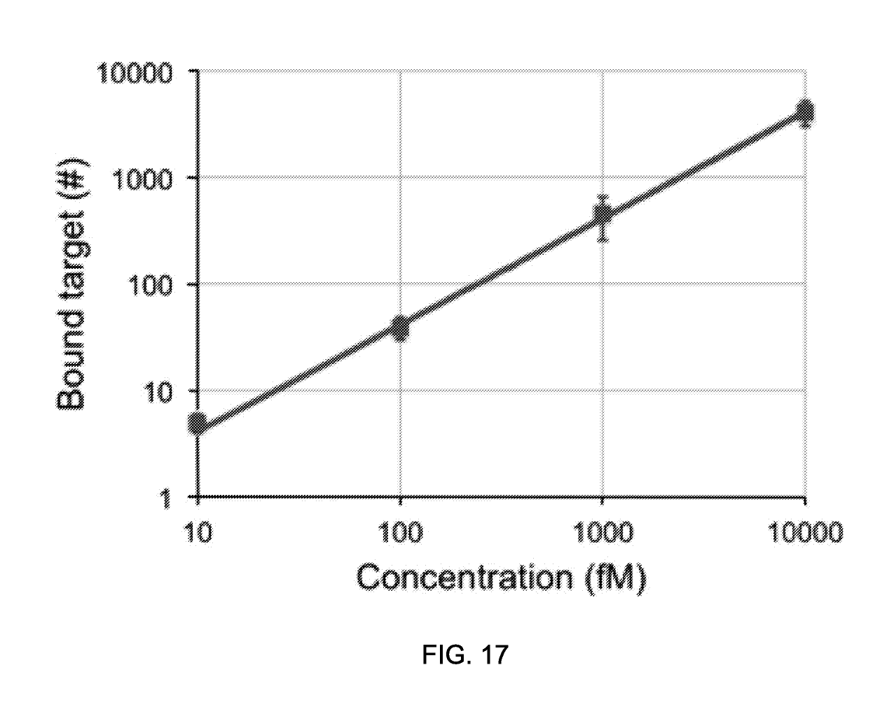

The detection sensitivity of the evaporating droplet microarray was tested by varying the target molecule (nucleic acid or protein) concentration from 10 fM to 100 pM. The bond QDs was quantified by using a custom Matlab program. As a control sample, one device area has hydrophilic islands pre-anchored with the scrambled probes, so any quantum dots left in those areas were due to incomplete wash or non-specific binding. We obtained the real binding events by subtracting the number of non-specifically bonded Q-dots from the detected events over the areas with DNA or ligand probes. The final results are shown in FIG. 17. A linear relationship between the streptavidin concentration and the number of streptavidin-QDs bonded to the biotin probes was obtained with the streptavidin concentration ranging from 10 fM to 10 pM. For higher target concentration beyond 10 pM, the bonding events were too dense to be resolved microscopically by the image processing program. For streptavidin concentration lower than 10 fM, the results were less reliable because the number of non-specific binding could be comparable with the number of specific binding. The amount of non-specific binding can be reduced by optimizing the washing conditions and proper surface treatments of the SiO₂ islands. Also the variation of the measurements can be further reduced by improved control of the droplet evaporation process through automation.

The Detection of miRNA Mimic Oligonucleotides

The sequence of anchor probe oligonucleotides is: 5′-TGCGA CCTCA GACTC CGGTG GAATG AAGGA AAAAA AAAAA-amine-3′. The target is miRNA 205 mimic oligonucleotides with a sequence of: 5′-TCCTT CATTC CACCG GAGTC TGAGG TCGCA-biotin-3′. miRNA 205 mimic was used here because miR205 has been reported as a specific biomarker for squamous cell lung carcinoma. The hybridization buffer (2% BSA, 50 mM borate buffer, 0.05% sodium azide, pH 8.3) was diluted 1000 fold before we spiked in the target oligonucleotides. Sample droplets (4 μL each) of different concentrations of miRNA 205 mimic oligonucleotides were pipetted to the black silicon template to form microdroplets. After the evaporation and oil encapsulation process described previously, the assay was incubated at 50° C. for hybridization. In the last step, streptavidin conjugated quantum dots (1 nM) was introduced to label those hybridized DNA duplex.

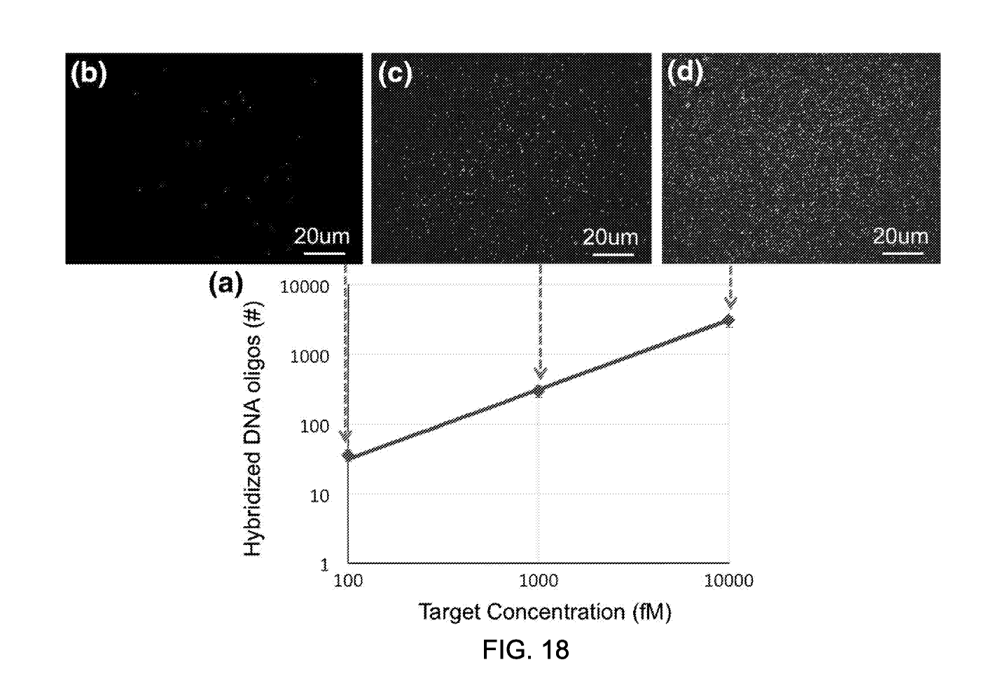

FIG. 18, panel (a) show the linear dependence of the number of hybridized targets and the concentration of target molecule in the sample. FIG. 18, panels (b-d) show the processed images of visualized quantum dots with a target concentration of 100 fM, 1 pM, and 10 pM, respectively. As shown in FIG. 18, panel (a), a linear relationship between the number of detected hybridized target and the DNA target concentration was obtained. However, the hybridization efficiency was found to be rather low (˜0.12%) and independent of the incubation time. The results indicated that the hybridization conditions within the oil encapsulated nanodroplet were not optimized. Clearly the hybridization process was no longer diffusion limited as it was in large reactors, and the likely reasons could be the non-ideal salt concentration and the density of DNA probes which may produce Coulomb repelling force to hinder the approach of DNA target molecules. Nonetheless we have achieved a sensitivity of 100 fM with a dynamic range of 2 orders of magnitude, which are among the best results demonstrated over a microarray platform. By optimizing the hybridization conditions such as the incubation temperature, DNA probe density, and salt concentration by varying the buffer dilution factor, the detention sensitivity is expected to be improved.

### APPLICATIONS

Disclosed is a novel oil-encapsulated nanodroplet array reactor for potential biosensing applications. The design has addressed the inherited slow, passive diffusion limitation commonly observed during DNA hybridization or protein—ligand binding by drastically decreasing the height of the reaction aqueous layer. Furthermore, the design greatly enriches the concentration of target molecules by several orders of magnitude in a controllable manner. Specifically, this enrichment procedure does not introduce amplification bias commonly found in thermal cycling or reverse transcription process (i.e., the enrichment factors for all the molecules are the same and independent of the GC contents of target DNAs). Hence, the disclosed technique can serve as a hybridization platform for direct detection of molecular markers of low abundance without requiring the enzymatic amplification process such as PCR, and offers a cost-effective, fast solution for point-of-care in vitro diagnosis.

The core technology for the oil-encapsulated evaporating droplet molecular detector platform is based on the fabrication of hydrophilic islands surrounded by a superhydrophobic surface. The superhydrophobic surface yields very large contact angle (˜160°) and eliminates the coffee ring effect by the receding boundary of the droplet. Black silicon was chosen to form the superhydrophobic surface because the formation of nanopillars that give black silicon its optical and surface properties is a self-forming process during deep reactive etching, avoiding the slow and expensive steps of fabricating nanopatterns over a large area.

Protocols have been developed to precisely control the evaporation process. Goniometer was used to closely monitor the evolution of the droplets during evaporation. Oil encapsulation terminated the evaporation process and formed a stable environment for the nanodroplet reactor without being affected by the outside environment such as humidity. Data has shown a detection sensitivity of 10 fM for streptavidin as a protein target and 100 fM for miRNA mimic oligonucleotides. A linear response was obtained for a concentration range spanning over 2 orders of magnitude. The detection sensitivity may be further enhanced by optimizing the hybridization conditions and reducing the diameters of hydrophilic islands. Furthermore, the device architecture can be easily scaled to increase the throughput and miniaturized footprint to support various molecular detection purposes desirable for point-of-care applications.

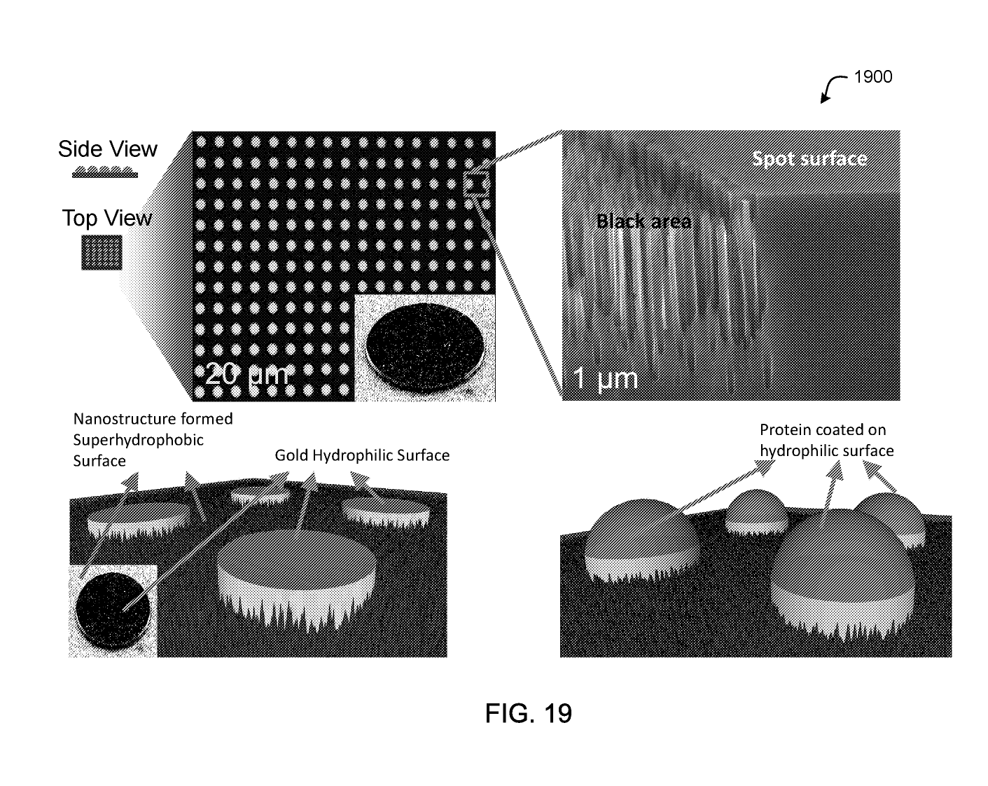

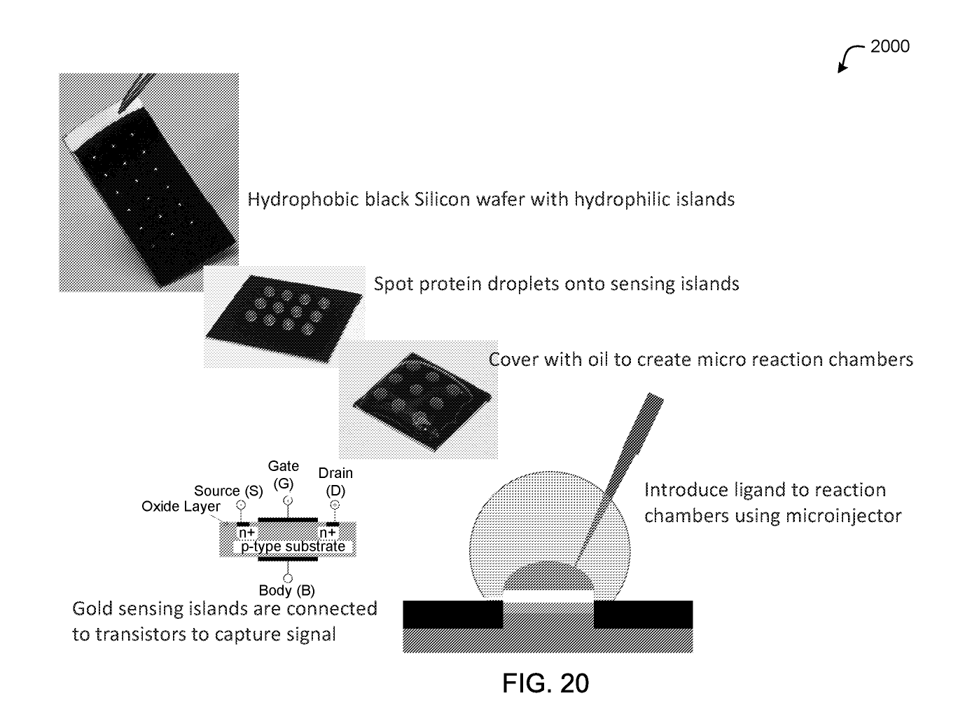

Other applications of the disclosed technology is possible. For example, FIG. 19 shows an exemplary high throughput drug screen chip 1900 implemented using the disclosed technology. Also, FIG. 20 shows an exemplary high throughput drug screen—silicon chip 2000 based platform implemented using the disclosed technology.

While this patent document contain many specifics, these should not be construed as limitations on the scope of any invention or of what may be claimed, but rather as descriptions of features that may be specific to particular embodiments of particular inventions. Certain features that are described in this patent document in the context of separate embodiments can also be implemented in combination in a single embodiment. Conversely, various features that are described in the context of a single embodiment can also be implemented in multiple embodiments separately or in any suitable subcombination. Moreover, although features may be described above as acting in certain combinations and even initially claimed as such, one or more features from a claimed combination can in some cases be excised from the combination, and the claimed combination may be directed to a subcombination or variation of a subcombination.

Similarly, while operations are depicted in the drawings in a particular order, this should not be understood as requiring that such operations be performed in the particular order shown or in sequential order, or that all illustrated operations be performed, to achieve desirable results. Moreover, the separation of various system components in the embodiments described in this patent document should not be understood as requiring such separation in all embodiments.

Only a few implementations and examples are described and other implementations, enhancements and variations can be made based on what is described and illustrated in this patent document.

What is claimed are techniques and structures as described and shown, including:

1. A device for characterization of single cellular entities, comprising:
a first substrate structured to include an array of hydrophilic regions surrounded by a hydrophobic surface including nanostructures protruding from the hydrophobic surface, wherein the array of hydrophilic regions is configured to adhere an individual cellular entity and the hydrophobic surface is configured to prevent the cellular entity from adherence; and
a second substrate including a coating of antibodies corresponding to a type of cellular substance secreted by the cellular entity,
wherein the second substrate is operable to be positioned on the first substrate such that the coating of antibodies of the second substrate makes contact with the individual cellular entities adhered to the hydrophilic regions of the first substrate.

2. The device of claim 1, wherein the cellular entity includes a cell, an organoid, or a tissue.

3. The device of claim 1, wherein the first substrate is shaped to insert within a well of a multi-well plate.

4. The device of claim 3, wherein the multi-well plate includes a 96-well plate.

5. The device of claim 1, wherein and the first substrate includes 1,000 or less hydrophilic regions in the array.

6. The device of claim 1, wherein the hydrophilic regions of the array include a dimension in a range of 50 μm to 400 μm.

7. The device of claim 1, wherein the hydrophilic regions include silicon oxide (SiO₂).

8. The device of claim 1, wherein the hydrophilic regions include a biocompatible material.

9. The device of claim 7, wherein the biocompatible material includes gold (Au) or Titanium (Ti).

10. The device of claim 1, wherein the hydrophilic regions are structured to include a layer forming an extracellular matrix (ECM) or a cellular seeding layer.

11. The device of claim 1, wherein the nanostructures include vertically aligned nanostructures including one or more of nanopillars, nanoposts, or nanopins having a diameter of 500 nm or less and a height of 6 μm or less.

12. The device of claim 1, wherein the second substrate includes fiducial markers in an arrangement to provide a point of reference or measurement for an image of the cellular substance bound to the coating.

13. The device of claim 1, wherein the cellular substance includes exosomes, signaling proteins, or cytokines.

14. The device of claim 1, wherein the hydrophilic regions include micro-islands and the hydrophobic surface includes black silicon.

15. The device of claim 1, wherein the hydrophobic surface has a contact angle greater than 155 degrees.

16. A method of fabricating a template for characterization of single cellular entities, the method including:
disposing hydrophilic wells over a substrate; and
disposing hydrophobic nanostructures surrounding the hydrophilic wells.

17. The method of claim 16, wherein the hydrophilic wells and the hydrophobic nanostructures are formed using separate etch masks.

18. The method of claim 16, wherein the hydrophobic nanostructures include black silicon.

19. The method of claim 16, wherein the wells range from a few micrometers to over 100 μm.

20. The method of claim 16, wherein the substrate includes a transparent substrate.

21. The method of claim 20, wherein the substrate includes glass or quartz.

22. The method of claim 20, including disposing a layer of amorphous Si on the transparent substrate.

23. The method of claim 20, wherein the substrate includes a silicon-on-sapphire wafer.
